(19) United States
(12) Patent Application Publication
(10) Pub. No.: US 2022/0192861 A1
(43) Pub. Date: Jun. 23, 2022

(54) MALE EXTERNAL CATHETER

(71) Applicant: Venkata Buddharaju, Park Ridge, IL (US)

(72) Inventor: Venkata Buddharaju, Park Ridge, IL (US)

(21) Appl. No.: 17/317,457

(22) Filed: May 11, 2021

Related U.S. Application Data
(63) Continuation-in-part of application No. 17/132,842, filed on Dec. 23, 2020.

(51) Int. Cl. A61F 5/453; A61F 5/44

(52) U.S. Cl. CPC A61F 5/453; A61F 5/4408

(57) ABSTRACT

A male external catheter includes a tubular body, a flexible flange at the first end of the tubular body surrounding the first opening and including a first side portion and a second side portion, and first and second strap portions including first and second strap ends, respectively. When the first and second strap ends are disconnected, the first and second strap portions do not apply sufficient force to the first and second side portions of the flange, respectively, to deform a shape of the first opening and the first opening has a first width. When the first and second strap ends are connected, the first and second strap portions apply sufficient force to the first and second side portions of the flange, respectively, to deform a shape of the first opening and the first opening has a second width narrower than the first width.

### CROSS REFERENCE

The present application comprises a continuation-in-part of U.S. application Ser. No. 17/132,842 filed Dec. 23, 2020, the entire contents of which are being incorporated herein by reference.

### BACKGROUND

The present disclosure relates to male external catheters. More specifically, the present disclosure is directed to a male external catheter including a flexible flange on a tubular body having varying wall thicknesses, wherein the flexible flange is deformed to provide a secure fit during use.

Condom catheters are alternatives to indwelling catheters that are less invasive to the patient. A conventional condom catheter is a sheath worn about the penis attached to tubing that directs urine to a drainage bag. The sheath is typically made of silicone and/or latex and includes a uniform wall thickness throughout the height.

An adhesive is typically provided on the interior of the opening of the sheath to adhere the condom catheter to the user's penis. In some catheters, a loop and pile (Velcro™) tape releaseable fastener or other fastener may wrap around the body of the catheter near the base thereof to secure the catheter about the penis.

Users of conventional condom catheters often experience pain or discomfort during use. The latex material may irritate the user's skin, and users often cannot anticipate having a reaction until the catheter is applied to the penis. Further, adhesives and other elements used to secure the catheter in place on the penis often get caught on the user's sensitive skin and hair within the region, leading to more acute pain. The adhesive material often times attaches too hard and tight around the penile skin, causing swelling, skin damage, and discomfort during use and/or removal. When left in place for too long, localized skin removal and/or wound formation may result, causing the patient to require the insertion of an indwelling catheter into the urethra and bladder for urinary drainage. Indwelling catheters increase the risk for urinary tract infections.

Another significant problem faced by condom catheters is leakage as well as difficulty retaining the condom catheter in place. Prior art systems including mechanisms to secure the condom catheter in place, such as complex belts with straps and/or adhesives. Belt systems tend to be cumbersome and uncomfortable, and do not ensure that leakage is avoided. Similarly adhesives may include gaps formed during application through which urine escapes. When urine and sweat interact with the adhesives, which is especially common in the pubic area having hair, the adhesive can become dislodged, resulting in leakage and needing to be replaced frequently, causing further pain and discomfort as well as increased health care costs.

Accordingly, there is a need for a male external catheter that minimizes leakage without utilizing adhesives or other additional components, as described herein.

### SUMMARY

To meet the needs noted above and others, the present disclosure provides a male external catheter that conforms or adapts to the size and fit around the user's penis as urine flows through, thereby maintaining its positioning without the need for adhesives or other securing mechanisms while reducing the likelihood of leakage. The male external catheter includes a flexible flange surrounding an opening into the body of the catheter, and a force is applied to the flange during use that deforms a shape of the opening to provide a secure hold on the user's penis. The presently claimed male external catheter provides an improved hold on the penis by uniformly exerting circumferential force around the penis while remaining soft on the skin so as to improve patient comfort. The male external catheter described herein is reusable, reducing waste as well as healthcare costs.

The body of the male external catheter has a tubular shape with a first end having a first opening through which the penis extends during use. A second end of the body has a second opening that connects to tubing of a drainage bag. A flexible flange at the first end of the tubular body surrounds the first opening and includes a first side portion and a second side portion on opposing sides of the flange. A first strap portion connected to the first side portion of the flange includes a first strap end, and a second strap portion connected to the second side portion of the flange includes a second strap end.

When the first and second strap ends are disconnected, the first and second strap portions do not apply sufficient force to the first and second side portions of the flange, respectively, to deform a shape of the first opening and the first opening has a first width. When the first and second strap ends are connected, the first and second strap portions apply sufficient force to the first and second side portions of the flange, respectively, to deform a shape of the first opening and the first opening has a second width narrower than the first width.

In one embodiment, the first and second side portions include first and second strap openings, respectively. The first and second strap portions extend through first and second strap openings, respectively, of the first and second side portions, respectively, of the flange.

During use, the first and second strap ends connect below the flange along a front of the body of the catheter. In one embodiment, the first and second strap portions are part of a single strap, and the strap includes a third portion between the first and second portions. The third portion extends below the flange along a back side of the body during use.

The flange comprises a flexible material that deforms under pressure. In some embodiments, the flange is a silicone material, although other suitable materials may be used as desired. The flange may be free from latex and/or adhesive.

A further benefit of the present design over the use of conventional catheters that utilize adhesives is the ability to temporarily allow airflow in through the flange of the catheter to allow the column of urine to drain from the tip of the catheter into the drainage bag. Without allowing air to enter the catheter, the column of urine remains in place between the catheter tip and the drainage bag, In the present design, the user can release the deformation of the shape of the opening by gently lifting the side portions of the flange against the bias provided by connected strap portions. This release allows airflow around the penis while the male external catheter is attached to the user, enabling the column of urine to drain into the drainage bag. The user can then allow the catheter to return to the deformed configuration, snugly fitting around the penis. In conventional adhesive catheters, the column of urine stays in contact with the skin and adhesive, risking the loss of adhesive by constant urine contact and causing softening of the skin and related skin loss. The adhesive catheter must be replaced altogether once the adhesive surface is separated from the skin, whereas the presently described male external catheter can be easily removed and washed for reuse.

In some embodiments, the body varies in cross-sectional area along a height thereof. The body includes a base portion, a central portion, and a tip portion, with the base portion being adjacent to the base and the tip portion configured to connect to tubing of a drainage bag. Each of the base portion, the central portion, and the tip portion include a base wall thickness, a central wall thickness, and a tip wall thickness, respectively. The central wall thickness is less than each of the base wall thickness and the tip wall thickness. This difference in wall thicknesses enables the central portion of the sheath to collapse around the user's penis while urine flows through the sheath and tip into the drainage bag.

The base portion of the body may be formed integrally with the flange. The base portion has a base height hb of approximately 0.830 in, although the base height hb may vary as desired or required during manufacturing. The base wall thickness t_{b} is preferably about 0.060 in at the greatest point, although the base wall thickness t_{b} may vary as desired or as required during manufacturing. In some embodiments, the base wall thickness t_{b} tapers from a predominant base wall thickness t_{b-p} to the central wall thickness t_{c} near the juncture of the base portion 102a and the central portion 102b. In one embodiment, the predominant base wall thickness t_{b-p} is about 0.060 in. In one embodiment, the base wall thickness tapers from a predominant base wall thickness to the central wall thickness near the juncture of the base portion and the central portion.

The central portion of the body includes a planar section adjacent to the base portion and a bellowed section adjacent to the tip portion. The bellowed section may include one or more horizontal bellows transverse to the height H of the body. In some embodiments, the planar section may include optional vertical ribbing leading up to and connected to the horizontal bellows. The vertical ribs may be evenly positioned along the perimeter of the body at or near the juncture of the planar section and the bellowed section. The central portion has a central height h_{c} of about 1.514 in, although the central height h_{c} may vary as desired or required during manufacturing. The central wall thickness t_{c} is preferably about 0.030 in, although the central wall thickness t_{c} may vary as desired or as required during manufacturing.

The tip portion of the body has a tip height hₜ of about 1.315 in, although the tip height hₜ may vary as desired or required during manufacturing. The tip wall thickness tₜ is preferably about 0.060 in although the tip wall thickness tₜ may vary as desired or required during manufacturing. In some embodiments, the tip portion includes horizontal ribbing along the perimeter of the cylindrical body.

During use, the user expands the opening in the flange and inserts the penis through the flange and into the body. The user then connects the first and second strap ends adjacent to the body near the flange such that sufficient pressure is applied to the first and second side portions of the flange to bend them towards the body of the catheter. Bending the first and second side portions of the flange causes the opening in the flange to deform from a first width to a narrower second width, which provides a more secure hold on the penis during use. Further, as urine flows through the tip portion of the body, the central portion of the body is pulled toward the penis and holds the user's penis as well, further securing the catheter on the penis and minimizing leakage out of the flange.

In light of the disclosure set forth herein, and without limiting the disclosure in any way, in a first aspect, which may be combined with any other aspect or portion thereof described herein, a male external catheter that connects to tubing of a drainage bag includes a tubular body, a flexible flange, and first and second strap portions. The body includes a first end having a first opening and a second end having a second opening. The flange at the first end of the tubular body surrounds the first opening and includes a first side portion and a second side portion on opposing sides of the flange. The first and second strap portions are connected to the first and second side portions of the flange, respectively, and include first and second strap ends, respectively. When the first and second strap ends are disconnected, the first and second strap portions do not apply sufficient force to the first and second side portions of the flange, respectively, to deform a shape of the first opening and the first opening has a first width. When the first and second strap ends are connected, the first and second strap portions apply sufficient force to the first and second side portions of the flange, respectively, to deform a shape of the first opening and the first opening has a second width narrower than the first width.

In a second aspect, which may be combined with any other aspect or portion thereof described herein unless otherwise specified, each of the first and second side portions of the flange includes first and second strap openings, respectively, and the first and second strap ends extend through first and second strap openings, respectively, of the first and second side portions, respectively, of the flange.

In a third aspect, which may be combined with any other aspect or portion thereof described herein unless otherwise specified, the male condom catheter includes first and second strap portions. The strap may also include a third portion that is positioned along the flange during use.

In a fourth aspect, which may be combined with any other aspect or portion thereof described herein unless otherwise specified, the flange comprises a silicone material. The flange may also be free from latex and/or adhesive.

In a fifth aspect, which may be combined with any other aspect or portion thereof described herein unless otherwise specified, the body includes a base portion adjacent to the flange and having a base wall thickness, a tip portion distal from the flange and including a tip configured to connect to the tubing of the drainage bag, wherein the tip portion has a tip wall thickness, and a central portion between the base portion and the tip portion and having a central wall thickness. The central wall thickness is less than at least one of the base wall thickness and the tip wall thickness. In a sixth aspect, which may be combined with any other aspect or portion thereof described herein unless otherwise specified, a method of using a male external catheter by a user is provided. The method comprises the steps of providing the male external catheter comprising a tubular body including a first end having a first opening and a second end having a second opening; a flexible flange at the first end of the tubular body surrounding the first opening and including a first side portion and a second side portion on opposing sides of the flange; a first strap portion connected to the first side portion of the flange, wherein the first strap portion includes a first strap end; and a second strap portion connected to the second side portion of the flange, wherein the second strap portion includes a second strap end. The method further includes the steps of expanding the first opening of the flange; inserting a penis of the user through the first opening and into the body; releasing the first opening of the base so that the male external catheter remains in position on the penis without the use of an adhesive; and connecting the first strap end and the second strap end such that sufficient force is applied to the first and second side portions of the flange, respectively, to deform a shape of the first opening.

In a seventh aspect, which may be combined with any other aspect or portion thereof described herein unless otherwise specified, the first strap end and the second strap end are connected below the flange.

In an eighth aspect, which may be combined with any other aspect or portion thereof described herein unless otherwise specified, the male external catheter further comprises a strap including the first strap portion and the second strap portion, and each of the first and second side portions of the flange includes first and second strap openings, respectively, and the method further comprises the step of positioning the strap through the first and second strap openings.

In a ninth aspect, which may be combined with any other aspect or portion thereof described herein unless otherwise specified, the strap further includes a third strap portion positioned below the flange on a side opposite of the connected first and second strap ends.

In a tenth aspect, which may be combined with any other aspect or portion thereof described herein unless otherwise specified, the method further comprises the step of inserting an object between the flange and the penis while the first strap end and the second strap end are connected to release negative pressure within the body of the male external catheter attached to the penis. In some embodiments, the object is a finger or tool.

In an eleventh aspect, any of the features, functionality and alternatives described in connection with any one or more of FIGS. 1 to 8B may be combined with any of the features, functionality and alternatives described in connection with any other of FIGS. 1 to 8B.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view from above of the male external catheter of the present application.

FIG. 2 is a perspective view from below of the male external catheter of FIG. 1.

FIG. 3 is a bottom plan view of the male external catheter of FIG. 1

FIG. 4 is a side elevational view of the male external catheter of FIG. 1.

FIG. 5 illustrates the male external catheter of FIG. 1 configured for a first method of use featuring a strap extending around the user's waist.

FIGS. 6A and 6B are a perspective view and a bottom plan view of the male external catheter of FIG. 1, respectively, of the present application, configured for a second method of use featuring disconnected strap ends adjacent to the catheter.

FIGS. 7A and 7B are a perspective view and a bottom plan view of the male external catheter of FIG. 1, respectively, of the present application configured for a second method of use featuring connected strap ends adjacent to the catheter.

FIGS. 8A and 8B are bottom plan and side elevational views, respectively, of the male external catheter of FIG. 1, illustrating dimensions.

### DETAILED DESCRIPTION

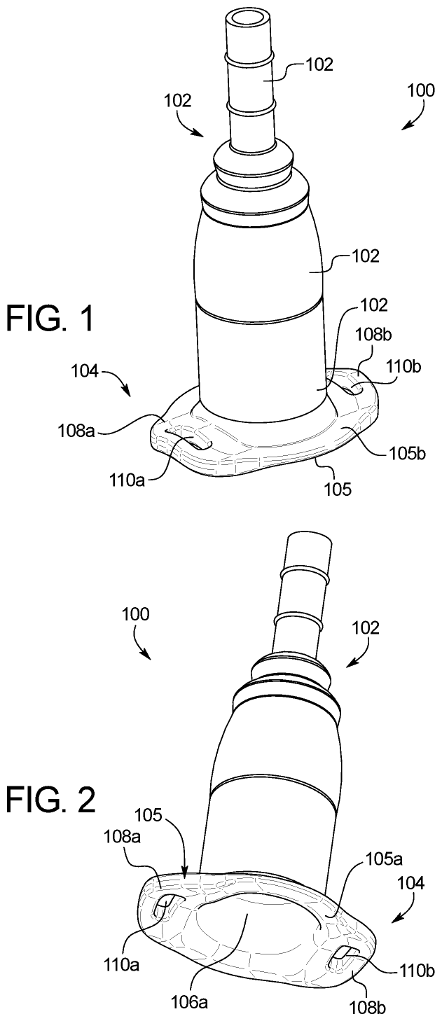

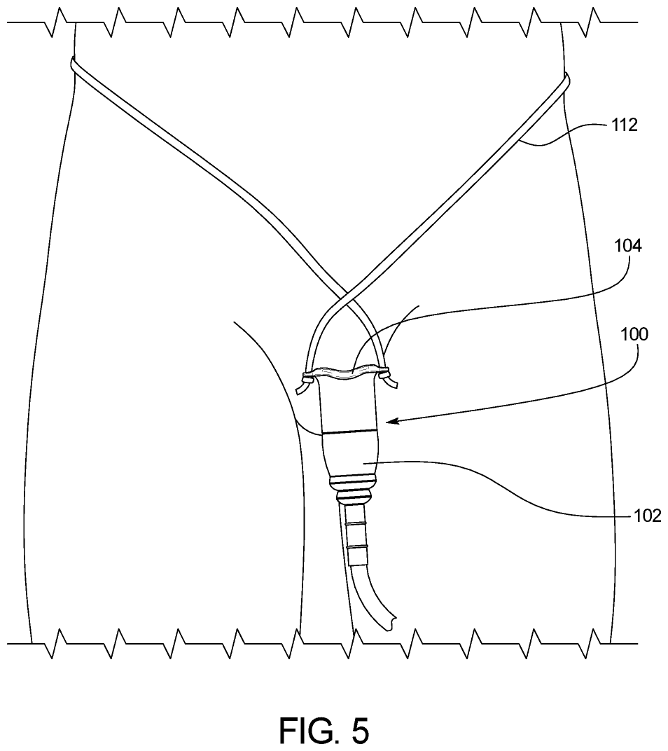

FIGS. 1-8B illustrate an example of a male external catheter 100 according to the present disclosure. Referring to FIGS. 1 and 2, the male external catheter or condom catheter 100 includes a body 102 having a tubular shape that extends from a flexible flange or base 104. The flange 104 is a body 105 providing a frame or structural support for a first opening 106a at a first end 102a of the body 102 through which the penis extends during use, as shown in FIG. 5. The tubular shape of the body 102 of the male external catheter 100 varies in cross-section along a height H (FIG. 4) thereof. The body 102 also includes a second opening 106b at a second end 102b for connection to tubing of a drainage bag.

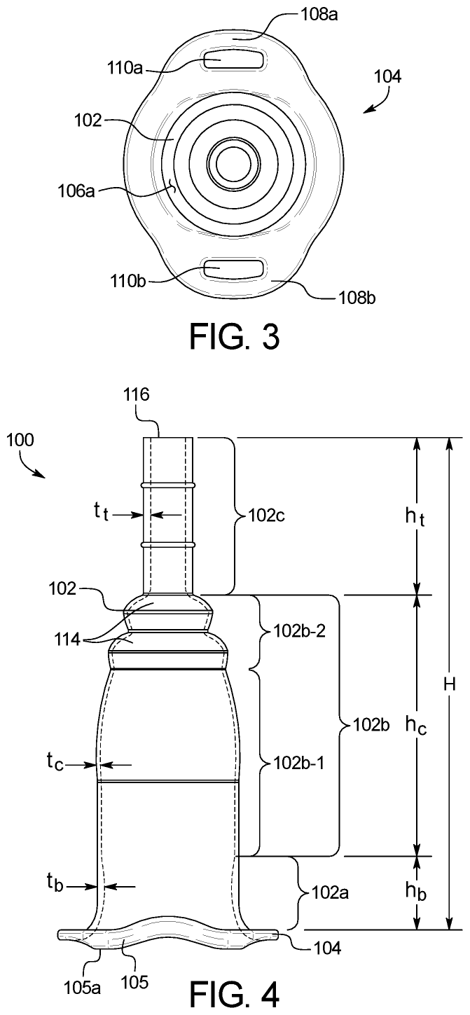

Referring to FIGS. 2 and 3, the opening 106a on the flange 104 of the male external catheter 100 defines an expandable sphincter that changes in size so that the opening 106a may conform to a diameter of a penis of the patient. The expandable sphincter provides a near liquid-tight seal between the male external catheter 100 and the penis so that urine may not easily escape. The expandable nature of the sphincter may also provide a comfortable fit for the patient and reduce the incidence of skin irritation as the body 102 of the condom catheter 100 does not slide or substantially shift when positioned on the penis.

As shown in FIG. 3, the flange 104 includes first and second side portions 108a, 108b on opposite sides of the opening 106a. Each side portion 108a, 108b has strap opening 110a, 110b for receiving a strap 112, 113 (FIGS. 5-7B). In some embodiments, the strap openings 110a, 110b have a slot shape.

During one example method of use illustrated in FIG. 5, a strap 112 may be fixedly or removably attached to the strap openings 110a, 110b of the base 104 and extend around the waist of the user to reinforce the positioning of the male external catheter 100 on the patient's body.

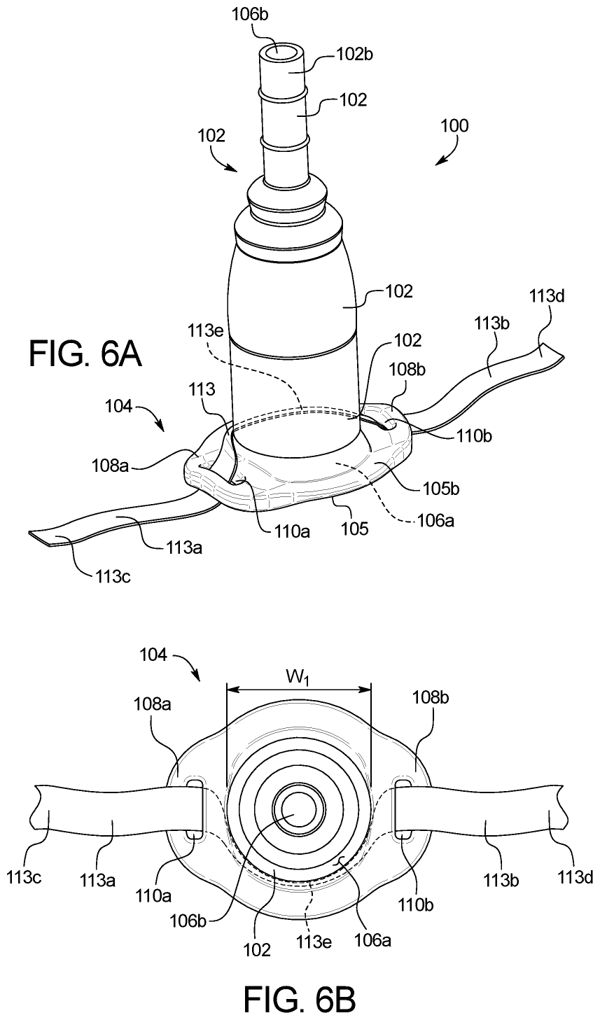

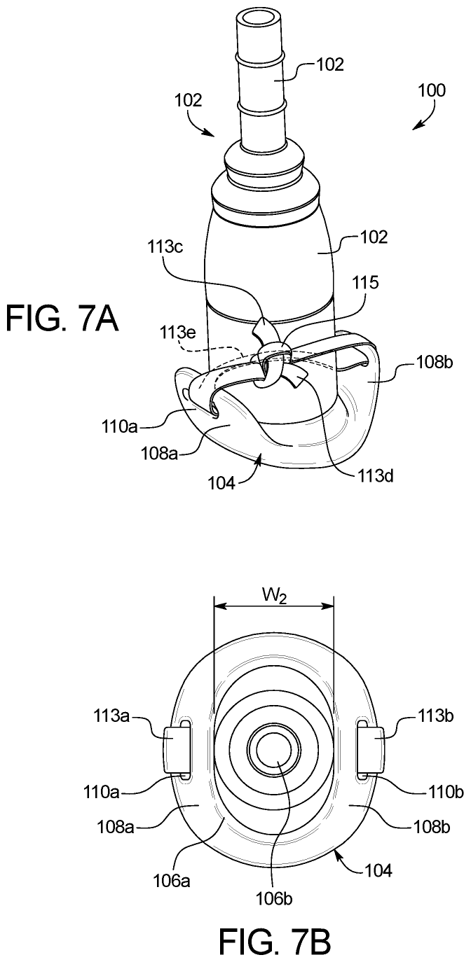

In another example method of use illustrated in FIGS. 6A-7B, a strap 113 may be fixedly or removably attached to the strap openings 110a, 110b of the flange 104 and connected together near the flange 104 so as to apply sufficient force to the first and second side portions 108a, 108b of the flange 104, respectively, to deform a shape of the opening 106a. In this embodiment, the strap 113 includes a first strap portion 113a and a second strap portion 113b connected to the first and second side portions 108a, 108b of the flange 104, respectively. The first and second strap portions 113a, 113b, respectively, include first and second strap ends 113c, 113d.

Referring to FIGS. 6A and 6B, the first and second strap ends 113c, 113d are initially disconnected such that the first and second strap portions 113a, 113b do not apply sufficient force to the first and second side portions 108a, 108b of the flange 104, respectively. The opening 106a, as a result, is not deformed and defines a first width W₁ of the opening 106a when in an undeformed configuration.

The opening 106a of the flange 104 of the male external catheter 100 is then stretched open and the penis is then inserted into the stretched, enlarged opening 106a, as described in greater detail below.

Once the male external catheter 100 is positioned on the user, the first and second strap ends 113c, 113d are connected at a point 115 as shown in FIGS. 7A and 7B such that the first and second strap portions 113a, 113b apply sufficient force to the first and second side portions 108a, 108b of the flange 104, respectively, to deform the shape of the opening 106a. Specifically, the first and second strap ends 113c, 113d connect together and cause the first and second side portions 108a, 108b of the flange 104 to bend towards the body 102 of the catheter 100, causing the opening 106a to move into a deformed configuration seen in FIG. 7B. When the strap ends 113c, 113d are connected, the deformed configuration of the opening 106a has a second width W₂ that is narrower than the first width W₁ of the undeformed configuration.

In the embodiment illustrated in FIGS. 6A-7B, the strap 113 is threaded through the strap openings 110a, 110b such that a third strap portion 113e between the first and second strap portions 113a, 113b extends below the flange 104 between the side portions 108a, 108b. During use, the third strap portion 113e extends below the flange 104 on a side opposite of the connected strap ends 113c, 113d. The third strap portion 113e provides additional reinforcement to tightly secure the opening 106a about the user's penis to prevent leaks.

When connecting the first and second strap ends 113c, 113d, the amount of tension needed is minimal in that the goal is to simply bend the side portions 108a, 108b of the flange 104 toward the body 102 of the catheter 100. Connecting the strap ends 113c, 113d too tightly and/or at a point 115 that is too close to the flange 104 may cause excessive deformation to the opening 106a and/or body 102, resulting in discomfort to the user. The amount of tension/force should be at least a minimum amount to cause the side portions 108a, 108b to bend towards the body 102 but less than a maximum amount to avoid excessive deformation and discomfort. Additionally, the first and second strap ends 113c, 113d are connected together below the flange 104 to avoid contact with the skin of the user.

After the strap ends 113c, 113d are connected, the flange 104 can be separated from the penile skin without much pressure. During use, urine flows from the body 102 through tubing into the drainage bag. A urine column forms between the body 102 of the catheter 100 and the drainage bag, and remains in place due to the negative pressure within the male external catheter 100. The user may briefly separate the flange 104 from the penile skin to allow airflow into the body 102 of the catheter 100, releasing the negative pressure therein and allowing urine within the tubing to drain into the drainage bag. The user can then allow the flange 104 to return to the deformed configuration, snugly fitting around the penis.

In other embodiments, the first and second strap portions 113a, 113b may be discrete components. Each strap portion 113c, 113d, 113d may be formed integrally with the respective side portion 108 of the flange 104 or may be connected through a hole and peg, an adhesive, or another suitable connection means.

In some embodiments, the strap 112, 113 may comprise an elastic, non-latex, and/or silicone material. In one example, the strap 113 comprises an elastic, non-latex material having a width of about 0.5 in and a length of about 12 in. In other embodiments, the strap 112, 113 may comprise an inelastic material. In still further embodiments, the strap 113 may comprise an elastic or inelastic material that connects together through a hook and loop fastener, an adhesive, or another suitable means of connection.

Referring to FIGS. 1 and 2, the body 105 includes a first surface 105a and a second surface 105b opposite the first surface 105a. In the illustrated embodiment, the first surface 105a is contoured to improve comfort and fit during use. The contoured first surface 105a is slightly concave. The body 102 of the male external catheter 100 extends from the second surface 105b, with the tubular shape thereof formed around the opening 106a of the flange 104.

Referring to FIG. 4, the body 102 includes a base portion 102a, a central portion 102b, and a tip portion 102c. The base portion 102a is adjacent to the base 104 and the tip portion 102c is distal from the base 104. The tip portion 102c is configured to connect to tubing of a drainage bag.

Each of the base portion 102a, the central portion 102b, and the tip portion 102c include a base wall thickness t_{b}, a central wall thickness t_{c}, and a tip wall thickness tₜ, respectively. In the illustrated embodiment, the central wall thickness t_{c} is less than each of the base wall thickness t_{b} and the tip wall thickness tₜ. In some embodiments, the central wall thickness t_{c} is less than one of the base wall thickness t_{b} and the tip wall thickness tₜ. This difference in wall thicknesses enables the central portion 102b of the body 102 to collapse around the user's penis while urine flows through the tip portion 102c and tip into the drainage bag.

In some embodiments, the ratio of the central wall thickness t_{c} to one or both of the base wall thickness t_{b} and the tip wall thickness tₜ is at least 1:1.1, preferably 1:1.2. In other embodiments, the ratio of the central wall thickness t_{c} to one or both of the base wall thickness t_{b} and the tip wall thickness tₜ is at least 1:1.5, and in still further embodiments, the ratio of the central wall thickness t_{c} to one or both of the base wall thickness t_{b} and the tip wall thickness tris at least 1:2.

The base portion 102a of the body 102 may be formed integrally with the base 104 and has a base height hb of approximately 0.830 in, although the base height hb may vary as desired or required during manufacturing. In one embodiment, the base wall thickness t_{b} is preferably about 0.060 in at the greatest point, although the base wall thickness t_{b} may vary as desired or as required during manufacturing. In one embodiment, the base wall thickness t_{b} tapers from a predominant base wall thickness t_{b-p} to the central wall thickness t_{c} near the juncture of the base portion 102a and the central portion 102b. In one embodiment, the predominant base wall thickness t_{b-p} is about 0.060 in.

The central portion 102b of the body 102 includes a planar section 102b-1 adjacent the base portion 102a and a bellowed section 102b-2 adjacent the tip portion 102c. The bellowed section 102b-2 includes horizontal bellows 114 transverse to the height H of the body 102, and the planar section 102b-1 may include optional vertical ribbing 116 connected to the horizontal bellows 114. The vertical ribs 116 may be evenly positioned along the perimeter of the body 102 near the juncture of the planar section 102b-1 and the bellowed section 102b-2.

In one embodiment, the central portion 102b has a central height h_{c} of about 1.514 in, although the central height h_{c} may vary as desired or required during manufacturing. The central wall thickness t_{c} is preferably about 0.030 in, although the central wall thickness t_{c} may vary as desired or as required during manufacturing.

The tip portion 102c of the body 102 has a tip height hₜ of about 1.315 in, although the tip height hₜ may vary as desired or required during manufacturing. The tip wall thickness tₜ is preferably about 0.060 in although the tip wall thickness tₜ may vary as desired or required during manufacturing. In some embodiments, the tip portion includes horizontal ribbing along the perimeter of the cylindrical body.

Shown in FIG. 4, the height H of the male external catheter 100 extends from the first surface 105a of the base 104 to a tip end surface 116.

The male external catheter 100 of the present application may come in different sizes to accommodate differently sized patients. While the dimensions of the structure may vary, the base wall thickness, the central wall thickness, and the tip wall thickness remain the same. In other words, while the diameter of the body may vary between sizes, the central wall thickness is preferably about 0.030 in while the base wall thickness and the tip wall thickness is about 0.060 in.

In the embodiment illustrated in FIGS. 1-7B, the male external catheter 100 may have the dimensions recited in the following table in reference to FIGS. 6A and 6B for a small-medium size and a medium-large size. It is understood that the dimensions are exemplary only and do not limit the scope of any claims herein, except as may be recited thereby, together with equivalents thereof. The dimensions may vary depending during the manufacturing process or as otherwise desired.

TABLE 1

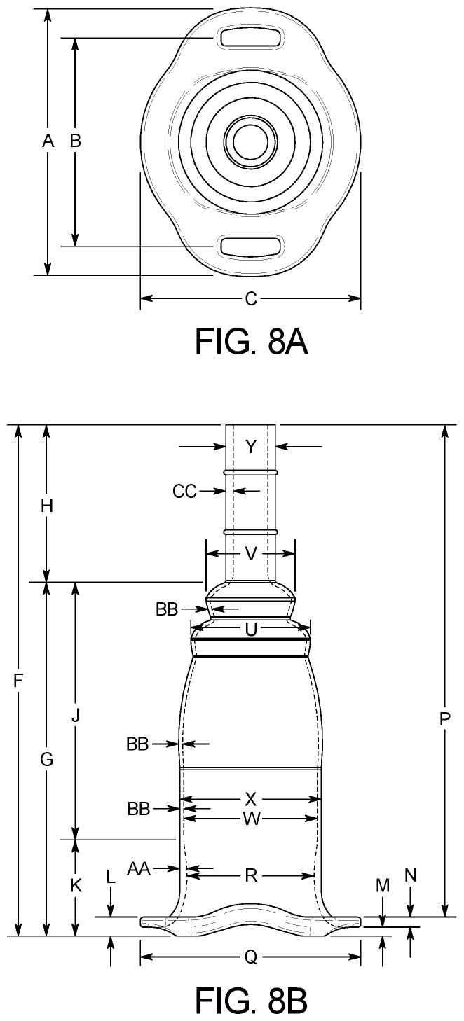

Dimensions (in)

| | Small-Medium Size | Medium-Large Size |
|---|---|---|
| FIG. 8A | | |
| A | 2.250 | 2.250 |
| B | 1.737 | 1.737 |
| C | 1.842 | 1.842 |
| FIG. 8B | | |
| F | 4.280 | 4.280 |
| G | 2.965 | 2.965 |
| H | 1.315 | 1.315 |
| J | 1.514 | 1.514 |
| K | 0.830 | 0.830 |
| L | 0.260 | 0.160 |
| M | 0.100 | 0.080 |
| N | 0.160 | 0.080 |
| P | 4.280 | 4.280 |
| Q | 1.842 | 1.607 |
| R | 0.827 | 1.062 |
| U | 1.003 | 1.003 |
| V | 0.747 | 0.747 |
| W | 0.887 | 1.122 |
| X | 0.947 | 1.182 |
| Y | 0.410 | 0.410 |
| AA | 0.060 | 0.060 |
| BB | 0.030 | 0.030 |
| CC | 0.060 | 0.060 |

In one embodiment, the male external catheter 100 is made of a silicone rubber compound, although other suitable materials may be used. The silicone rubber compound may be a peroxide-cured silicone elastomer including polydimethylsiloxane with vinyl functional groups. In one embodiment, the compound comprises approximately 70% to approximately 90% by weight polydimethylsiloxane. The silicone rubber material may be hardness (Shore A hardness test) of 20 points, a tensile strength of 800 psi, an elongation at rupture of 800%, and a specific gravity of 1.2. In some embodiments, the male external catheter 100 is latex free. In other embodiments, the body 102 and the flange 104 may be composed of the same material or different materials.

During use of the device as shown in FIG. 5, the patient is placed in a supine position and the elastic strap is positioned under the lower back of the patient. The opening 106a of the flange 104 of the male external catheter 100 is stretched open using index and middle fingers. The height H of the male external catheter 100 is compressed, using thumbs to push the tip portion 102c of the body 102 toward the base 104. The penis is then inserted into the stretched, enlarged opening 106a, and the base portion 102a of the body 102 is pulled and pushed over and above the glans penis towards the base of the penis as far as possible.

A first end of the elastic strap 112 is extended from a first side of the patient across the lower abdomen and secured to the side portion 108 opposite the first side of the patient. A second end of the elastic strap 112 is then extended from a second side of the patient across the lower abdomen and secured to the side portion 108 opposite the second side of the patient.

Referring to use of the device as shown in FIGS. 6A-7B, the patient is placed in a supine position. The opening 106a of the base 104 of the male external catheter 100 is stretched open using index and middle fingers. The height H of the male external catheter 100 is compressed, using thumbs to push the tip portion 102c of the body 102 toward the base 104. The penis is then inserted into the stretched, enlarged opening 106a, and the base portion 102a of the body 102 is pulled and pushed over and above the glans penis towards the base of the penis as far as possible.

The first end 113c and the second end 113d of the strap 113 are drawn together in front of the body 102 below the flange 104 and tied such that sufficient force is applied to the first and second side portions 108a, 108b of the flange 104, respectively, to deform a shape of the opening 106a.

In particular, the deformation of the opening 106a from the first width W₁ to the second width W₂ combined with the variation in wall thickness of the body 102 provides a male external catheter that maintains positioning on the user's penis with minimal to no leakage and without undue pressure on the penis during use. Use of the external male catheter 100 as shown in FIGS. 6A-7B eliminates the use of a strap around the body as shown in FIG. 5 as well as the use of adhesives, foam straps, or additional straps that often lead to slippage, frequency changes, and skin erosion and damage.

Further, a negative pressure exists within the body 102 of the male external catheter 100 is being used to drain urine to the drainage bag such that urine remains in the tubing between the male external catheter 100 and the drainage back. A tool, a finger, or another object may be used to separate the flange 104 from the penis of the user during use to release the negative pressure. This release of negative pressure allows the urine to flow freely through the tubing to the drainage bag, away from the male external catheter 100.

It should be noted that various changes and modifications to the presently preferred embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein

1. A male external catheter that connects to tubing of a drainage bag, the male external catheter comprising:
a tubular body including a first end having a first opening and a second end having a second opening;
a flexible flange at the first end of the tubular body surrounding the first opening and including a first side portion and a second side portion on opposing sides of the first opening, wherein the first opening is configured to move between an undeformed configuration and a deformed configuration, the first opening having a first width in the undeformed configuration and a second width in the deformed configuration, the first width being greater than the second width;
a first strap portion connected to the first side portion of the flexible flange; and
a second strap portion connected to the second side portion of the flexible flange;
wherein the first strap portion and the second strap portion are connected to each other and apply a force to the first and second side portions, respectively, of the flexible flange sufficient to maintain the first opening in the deformed configuration.

2. The male external catheter of claim 1, wherein the first and second side portions of the flexible flange include first and second strap openings, respectively, and the first and second strap portions include first and second strap ends, respectively, each configured to extend through the first and second strap openings, respectively, of the first and second side portions, respectively, of the flexible flange.

3. The male external catheter of claim 2, further comprising a strap including the first strap portion, the second strap portion, and a third strap portion, wherein the third strap portion is positioned adjacent the flexible flange around the tubular body.

4. The male external catheter of claim 1, wherein the flexible flange comprises a silicone material.

5. The male external catheter of claim 4, wherein the flexible flange is free from latex.

6. The male external catheter of claim 4, wherein the flexible flange is free from adhesive.

7. The male external catheter of claim 1, wherein the tubular body includes:
a base portion adjacent to the flange and having a base wall thickness;
a tip portion distal from the flange and including a tip configured to connect to the tubing of the drainage bag, wherein the tip portion has a tip wall thickness; and
a central portion between the base portion and the tip portion and having a central wall thickness;
wherein the central wall thickness is less than at least one of the base wall thickness and the tip wall thickness.

8. A method of using a male external catheter by a user, the method comprising the steps of:
providing the male external catheter comprising:
a tubular body including a first end having a first opening and a second end having a second opening;
a flexible flange at the first end of the tubular body surrounding the first opening and including on opposing sides of the first opening, wherein the first opening is configured to move between an undeformed configuration and a deformed configuration;
a first strap portion connected to the first side portion of the flexible flange; and
a second strap portion connected to the second side portion of the flexible flange;
wherein, when the first and second strap portions are disconnected from each other, the first opening is biased toward the undeformed configuration having a first width; and
wherein, when the first and second strap portions are connected to each other in a position to apply a sufficient force to the first and second side portions of the flexible flange, respectively, the first opening moves into the deformed configuration having a second width narrower than the first width;
expanding the first opening of the tubular body;
inserting a penis of the user through the first opening and into the tubular body;
releasing the first opening of the tubular body such that the first opening is positioned on the penis without the use of adhesive, the first opening being provided in the undeformed configuration having the first width; and
pulling the first strap portion and the second strap portion to each other in a position to apply a sufficient force to the first and second side portions of the flexible flange, to cause the first opening to move into the deformed configuration having the second width narrower than the first width in order to secure the first opening snugly on the penis of the user without the use of adhesive; and
connecting the first and second strap portions together.

9. The method of claim 8, wherein the first strap portion and the second strap portion are connected below the flexible flange.

10. The method of claim 8, wherein the male external catheter further comprises a strap including the first strap portion and the second strap portion; wherein each of the first and second side portions of the flexible flange includes first and second strap openings, respectively; and wherein the method further comprises the step of positioning the strap through the first and second strap openings.

11. The method of claim 10, wherein the strap further includes a third strap portion positioned around the tubular body on a side opposite of the connected first and second strap portions.

12. (canceled)
